(19) United States
(12) Patent Application Publication
(10) Pub. No.: US 2012/0237994 A1
(43) Pub. Date: Sep. 20, 2012

(54) BIOCHARS, METHODS OF USING BIOCHARS, METHODS OF MAKING BIOCHARS AND REACTORS

(75) Inventors: Keshav C. Das, Athens, GA (US); Nagamani Balagurusamy, Torreon (MX); Senthil Chinnasamy, Chennai (IN)

(73) Assignee: University of Georia Research Foundation, Inc., Athens, GA (US)

(21) Appl. No.: 13/388,907

(22) PCT Filed: Aug. 12, 2010
(86) PCT No.: PCT/US10/45266

§ 371 Date: Jun. 8, 2012

Related U.S. Application Data
(60) Provisional application No. 61/233,234, filed on Aug. 12, 2009.

(51) Int. Cl. C12N 11/14; C12M 1/00; C12P 5/02

(52) U.S. Cl. 435/167; 435/176; 435/289.1

(57) ABSTRACT

Embodiments of the present disclosure provide for biochar impregnated with microbes, methods of making biochar impregnated with microbes, methods of using biochar impregnated with microbes, methods of using biochar to produce gas, reactors using biochar and/or biochar impregnated with microbes, methods of using the reactors, and the like.

### CROSS-REFERENCE TO RELATED APPLICATIONS

This application claims priority to U.S. provisional application entitled, “BIOCHARS, METHODS OF USING BIOCHARS, METHODS OF MAKING BIOCHARS, AND REACTORS,” having Ser. No. 61/233,234, filed on Aug. 12, 2009, which is entirely incorporated herein by reference.

### STATEMENT REGARDING FEDERALLY SPONSORED RESEARCH OR DEVELOPMENT

This invention(s) was made with government support awarded by the USAID, 523A00060000900. The government has certain rights in the invention(s).

### BACKGROUND

The use of biofuels reduces oil dependence and provides national security, environmental, and economical benefits. Biogas produced through anaerobic digestion (AD) is an important sector in the biofuels science. It is an attractive waste treatment practice in which both pollution control and energy recovery can be achieved. AD has seen a resurgence of interest in the recent past due to its potential for manure stabilization, sludge reduction, odor control, green energy production, and carbon credits. Carbon credits may have significant economic impact on AD profitability for the coming decades as the U.S. and other global economies use emerging greenhouse gas (GHG) offset markets. Even though AD has high standards of maintenance and management along with high initial capital investment, a properly functioning AD can provide numerous benefits such as: (1) odor control; (2) reduction of nuisance gas emissions; (3) potential pathogen kill; (4) reduction of wastewater strength (oxygen demand); (5) conversion of organic nitrogen into plant available ammonia nitrogen; (6) preservation of plant nutrients (e.g., N, P, K) for use as a high quality fertilizer; and/or (7) production of a renewable energy source-biogas.

Many agricultural, municipal, and industrial wastes are ideal candidates for AD because they contain high levels of easily biodegradable materials. AD is considered to be a low hanging fruit for converting biomass wastes into bioenergy. Though the technology is mature, it is not being widely followed due to high capital cost and operational problems such as shock loading, souring of reactors, and/or odor. Problems such as low methane yield and process instability are often encountered in anaerobic digestion.

Therefore there is a need to address at least these disadvantages.

### SUMMARY

Briefly described, embodiments of this disclosure include, among others, provide for biochar impregnated with microbes, methods of making biochar impregnated with microbes, methods of using biochar impregnated with microbes, methods of using biochar to produce gas, reactors using biochar and/or biochar impregnated with microbes, methods of using the reactors, and the like.

One exemplary material, among others, includes a biochar impregnated with at least one type of microbe (e.g., algae, fungi, bacteria, archaea, protists, and a combinaton thereof).

One exemplary method of producing a gas, among others, includes exposing a biochar described herein to a material selected from the group consisting of: a biomass, manure, and a combination thereof; and producing a gas from the interaction of the material with the microbes.

One exemplary reactor for producing a gas, among others, includes a reactor including a biochar as described herein.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a graph that illustrates cumulative methane production in 100% dairy manure.

FIG. 2 is a graph that illustrates cumulative methane production in 100% poultry litter.

FIG. 3 is a graph that illustrates the total biogas production in 75% dairy manure+25% algae mixture with and without char addition.

FIG. 4 is a graph that illustrates methane production in 75% dairy manure+25% algae mixture with and without char addition.

FIG. 5 is a graph that illustrates protein content on pine derived biochar with exposure to rumen inoculum.

FIGS. 6A to 6F illustrate scanning electron microscope pictures that show morphologies found on pine char surface at different incubation times.

FIG. 7 is a graph that illustrates biogas production using peanut biochar that is exposed to a sterile feedstock.

FIG. 8 is a graph that illustrates biogas production using peanut biochar that is exposed to a non-sterile feedstock

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of chemistry, organic chemistry, the agricultural sciences, biology, physics, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions:

In this specification and in the claims that follow, reference will be made to a number of terms that shall be defined to have the following meanings unless a contrary intention is apparent.

The terms “manure” or “animal manure” refer to feces. Common forms of animal manure include farmyard manure, farm slurry, poultry manure, cattle manure, swine manure, poultry litter, and the like. It often contains plant material (straw or wood shavings) that has been used as bedding for animals and has absorbed the feces and urine. It is often produced by intensive livestock rearing systems. Animal manures may also include other animal products, such as feathers, hairs, bones, blood, and dead animals.

“Biochar” is a carbonized form of a plant material that is specifically produced for non-fuel applications. The process of production gives biochar properties that make it suitable for applications such as adsorption (e.g., gas adsorption), enhancing microbial activity, and the like. Production processes can be batch or continuous, where base material of particle sizes ranging from a few millimeters to several centimeters are placed in a retort, with or without carrier gas flowing through. Carrier gases may be non-reactive such as nitrogen, or reactive such as steam. The retort may be heated by external heat or directly heated by combusting a portion of the base material. Vapors emanating may be captured for other applications. After a period of several minutes to hours, the residual material remaining is biochar. Biochar is composed of mainly carbon (about 30 to 100% or about 60 to 100%) and is porous (e.g., a material having numerous pore spaces of varying diameter and length (depth), where total void volume of material is about 50% or more). Other elements (such as nitrogen, oxygen, hydrogen) are gradually lost at elevated temperatures. The molecular structure and elemental composition makes biochar highly recalcitrant against microbial decomposition.

The term “biomass” can include biological material from organisms. “Biomass” can be created as products, by-products, and/or residues of the forestry and agriculture industries. Biomass includes, but is not limited to, plants, trees, crops, crop residues, grasses, forest and mill residues, wood and wood wastes, fast-growing trees, and combinations thereof. In addition, “biomass” can include or be created from microbes such as algae, bacteria, fungi, protest, archaea, and combinations thereof.

The term “microbe” can include algae, bacteria, archaea, protists, and/or fungi, while microbes can include a mixture thereof. The term “microbe(s)” can also include green algae, planarian, and/or plankton, or a mixture thereof.

The terms “bacteria” or “bacterium” include, but are not limited to, Gram positive and Gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M, ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The Gram-positive bacteria may include, but is not limited to, Gram positive Cocci (e.g., Streptococcus, Staphylococcus, and Enterococcus). The Gram-negative bacteria may include, but is not limited to, Gram negative rods (e.g., Bacteroidaceae, Enterobacteriaceae, Vibrionaceae, Pasteurellae and Pseudomonadaceae). In an embodiment, the bacteria can include Mycoplasma pneumoniae.

The term “protozoan” as used herein includes, without limitation, flagellates (e.g., Giardia lamblia), amoeboids (e.g., Entamoeba histolitica), and sporozoans (e.g., Plasmodium knowlesi) as well as ciliates (e.g., B. coli). Protozoan can include, but it is not limited to, Entamoeba coli, Entamoeabe histolitica, Iodoamoeba buetschlii, Chilomastix meslini, Trichomonas vaginalis, Pentatrichomonas homini, Plasmodium vivax, Leishmania braziliensis, Trypanosoma cruzi, Trypanosoma brucei, and Myxoporidia.

The term “algae” as used herein includes, without limitation, microalgae and filamentous algae such as Anacystis nidulans, Scenedesmus sp., Chlamydomonas sp., Clorella sp., Dunaliella sp., Euglena so., Prymnesium sp., Porphyridium sp., Synechoccus sp., Botryococcus braunii, Crypthecodinium cohnii, Cylindrotheca sp., Microcystis sp., Isochrysis sp., Monallanthus salina, M. minutum, Nannochloris sp., Nannochloropsis sp., Neochloris oleoabundans, Nitzschia sp., Phaeodactylum tricornutum, Schizochytrium sp., Senedesmus obliquus, and Tetraselmis sueica as well as algae belonging to any of Spirogyra, Cladophora, Vaucheria, Pithophora and Enteromorpha genera.

The term “fungi” as used herein includes, without limitation, a plurality of organisms such as molds, mildews and rusts and include species in the Penicillium, Aspergillus, Acremonium, Cladosporium, Fusarium, Mucor, Nerospora, Rhizopus, Tricophyton, Botryotinia, Phytophthora, Ophiostoma, Magnaporthe, Stachybotrys and Uredinalis genera.

The term “archaea” as used herein includes, without limitation, Crenarchaeota, Euryarchaeota, Korarchaeota, Nanoarchaeota, and Thaumarchaeota, as well as unclassified archaea.

### General Discussion

Embodiments of the present disclosure provide for biochar impregnated with microbes, methods of making biochar impregnated with microbes, methods of using biochar impregnated with microbes, methods of using biochar to produce gas, reactors using biochar and/or biochar impregnated with microbes, methods of using the reactors, and the like.

Embodiments of the present disclosure can enhance gas production, such as methane production, from biomass, livestock waste, food industry wastes, and other industrial wastes by using biochar, which acts as a stimulant and support medium for anaerobes to enhance the population of hydrolytic microbes such as bacteria and/or archaea. In addition, embodiments of the present disclosure include a biochar that helps in reducing the toxicity of various chemical components and protects methanogens since they can be disposed within the pores of the biochar.

Embodiments of the present disclosure can include a biochar impregnated with microbes. The terms “impregnate” or “impreganted” refer to the occupation of a portion (e.g., about 20, 40, 50, 60, 70, 80, 90, 99% or more of the space within the pore) of the porous structure of the biochar so that the microbes permeate the porous structure of the biochar, and then the microbes can interact with the materials (e.g., biomass, manure, etc) in the reactor. In an embodiment, the microbes can also be present on the surface of the biochar as well. The biochar can be impregnated with the microbes prior to addition to a reactor and/or the biochar can become impregnated in the biochar after introduction of the biochar to the reactor. In an embodiment, the microbes can include algae, bacteria, archaea, protists, fungi, and a combinaton thereof. In particular, the microbes can be bacteria and/or archaea.

In an embodiment the amount of microbes and/or the types of microbes can be dependent upon, for example, the microbes present in a biomass (or other material) introduced to the reactor, microbes present in the reactor, the material used to produce the biochar, the surface area of the biochar, conditions used in the reactor to produce gas, surface chemistry, porosity, pore size distribution, and the like. In general, the amount of microbes present in a quantitiy of biochar can be difficult to determine. In an effort to estimate the amount of microbes in a quanity of biochar, the amount of protein in an amount of biochar was determined. The amount of protein can be correlated with the amount of micorbes. An estimation of the amount of protein present for a quantity of biochar can be about 1000 to 2000 micrograms per 0.5 grams of biochar or about 1500 micrograms per 0.5 grams of biochar (See Examples). In an embodiment, the number of microbes is on the order of about 10¹² microbes per gram of char. This number was an estimate based on SEM pictures and assuming an average coverage of 25% of the biochar. Thus, the number of microbes can be estimated based on the average coverage of the biochar. The coverage of the biochar can be from about 1, about 10, about 20, about 30, about 40, or about 50, to about 99, about 90, about 80, about 70, about 60, or about 50, and any combination of these percentages.

In an embodiment, the microbes can be used to produce hydrocarbon gases such as methane, ethane, other similar hydrocarbon gases and combinations of these gases. In an embodiment, the microbes can include methanogens (e.g., bacteria, archaea, and the like) to produce methane gas. Methanogens can produce methane as a metabolic product or by-product. In the present embodiment, the methanogens can produce methane under conditions present in the reactors using the biochar and/or biochar impregnated with microbes (See, Examples).

Embodiments of the present disclosure provide for methods of producing a gas (e.g, hydrocarbon (e.g., methane) or a biogas (e.g., methane, carbon dioxide, other possible components such as nitrogen, hydrogen, hydrogen sulfide, and oxygen)) using biochar and/or biochar impregnated with microbes from a material such as, but not limited to, manure (e.g., livestock manure), decomposing material such as biomass (e.g., decomposing algal biomass), and a combination thereof. In an embodiment, the livestock manure is a material such as, but not limited to, poultry litter, dairy manure, swine manure, and a combination thereof In an embodiment, the biomass can include products, by-products, and/or residues of the forestry and agriculture industries or algal biomass or other biomass. In an embodiment, the material can be a mixture of one or more types of biomass and/or one or more types of manure. For example, the material can have a ratio of manure to biomass of about 1:100 to 100:1, and any increment of 1 there between (e.g., 1:4, 4:1, 3:10, 10:3, and the like). In another example, the material can have a ratio of one type of manure (or biomass) to a second type of manure (or biomass) of about 1:100 to 100:1, and any increment of 1 there between (See Examples for additional details). It should be noted that 3, 4, 5, or 10 different types of biomass and/or manure can be present in a material since in some instances the source of the material is a farm, pond, composting site, and the like.

In an embodiment, both a biomass and manure can be used in the reactor with an impregnated biochar. In an embodiment, both a biomass and manure can be used in the reactor with a biochar that is not impregnated. After introduction of the biochar, biomass, and the manure, the biochar can become impregnated with microbes from the biomass and/or from another source.

Embodiments of the present disclosure can include a biochar that can be impregnated with microbes such as methanogens (e.g., at high concentrations such as about 1 to 100 million microbes per mL, but this amount can vary depending on the microbes and percent coverage of the biochar) for use to directly microbially seed the anaeorobic digester reactor beds. The microbial population in the digester should be high and should remain relatively stable (e.g., reduce shock loads from, for example, pH changes) thus providing high performance (e.g., reduction of time to produce biogas, for example an embodiment of the present disclosure can reduce the time to produce biogas from about 21 days to 5 or 6 days) of digestion, and use of the biochar (e.g., impregnate and/or un-impregnated biochar) of the present disclosure can accomplish these goals. In addition, embodiments of the present disclosure provide for a low-cost reactor configuration to immobilize methanogens, for example, in anaerobic digesters by using biochar (e.g., that is impregnated with the methanogens) to overcome the problems of toxicity and acidity (both of which can reduce the population of the microbes). One or more of these solutions can help dairy, poultry, and swine farms as well as the food industry to generate income from their waste products.

Embodiments of the present disclosure include reactors including biochar and/or biochar impregnated with microbes. In an embodiment, the microbes are introduced to the reactor separately from the biochar. In this regard, the microbes may enter the biochar once inside the reactor (e.g., anaerobic digester). In another embodiment, the biochar can be impregnated with microbes prior to introduction to the reactor. In yet another embodiment, impregnated biochar can be introduced to the reactor and the impregnated biochar can be further impregnated with more microbes (same or different types). In yet another embodiment, both impregnated biochar and biochar can be introduced to the reactor.

Biochar impregnated with microbes can reduce the production time for equivalent amounts of biogas by about 60% or more, relative to using biochar not impregnated with microbes.

The reactor can also include a material such as a biomas, manure, or the like and other components (e.g., solvents, water, acids, bases, catalyst, and the like) to faciliate the production of a gas such as methane. The biochar can also adsorb gases for emission control (e.g., NH₃, H₂S) that are generated in the reactor. In addition, an embodiment of the present disclosure can have a chemical oxygen demand removal rate that are over about 40% and in some instances over 99%. Thus, in an embodiment, impregnated biochar and biochar can be introduced to the reactor to accomplish the production of gas (e.g., methane) and the absorption of other gases (e.g., NH₃).

The reactor can be a batch reactor or an upflow reactor known in the art. In general, the solids (biochar or impregnated biochar) content in the reactor can be about 1 to 10 weight % of the total mass of digestible material in the reactor. In some instances, the solids content in the reactor is about 1 to 2, about 2 to 6, about 2.5, or about 5, weight % of the total mass of digestible material in the reactor. The amount of biochar used in a reactor can depend, at least in part, upon the reactor type, the materials (e.g., biomass, manure, and combinations thereof), the temperature, components added to the reactor, the type and/or amount of microbes, the amount of gas desired to be produced, and the like.

Additional embodiments of the present disclosure are described in the Examples.

### EXAMPLES

Now having described the embodiments of the present disclosure, in general, the Examples describe some additional embodiments of the present disclosure. While embodiments of present disclosure are described in connection with the Examples and the corresponding text and figures, there is no intent to limit embodiments of the present disclosure to these descriptions. On the contrary, the intent is to cover all alternatives, modifications, and equivalents included within the spirit and scope of embodiments of the present disclosure.

### EXAMPLES

### Example 1

This portion describes the study of the effects of addition of biochar to enhance methane production from Poultry litter (PL) and Dairy manure (DM)

### Methodology

The study was performed in batch reactors of 2 L capacity with a working volume of 30%. Experiments were conducted for a period of 80 days at a constant temperature of 27° C. Different ratios of poultry litter and dairy manure viz. 100% poultry litter (PL), 75% (PL)+25% Dairy Manure (DM), 50% (PL)+50% (DM), 25% (PL)+75% (DM) and 100% (DM), were used in the study. All the reactors were inoculated with inoculum enriched in PL at 10% level and were amended with addition of charcoal at 2.5 and 5% concentrations. Reactors with all the mixtures without inoculum and amendment (biochar) addition were maintained as control. Total biogas production and methane concentration were analyzed for every four days by water displacement and GC, respectively. Volatile fatty acids were analyzed by using gas chromatography (FID). C, H, N and S were estimated by CHNS Analyzer (Leco, USA). Volatile solids were analyzed by proximate analyzer (Leco, USA). Total Solids were analyzed by following standard methods and chemical oxygen demand by using Hach test kits.

### Results

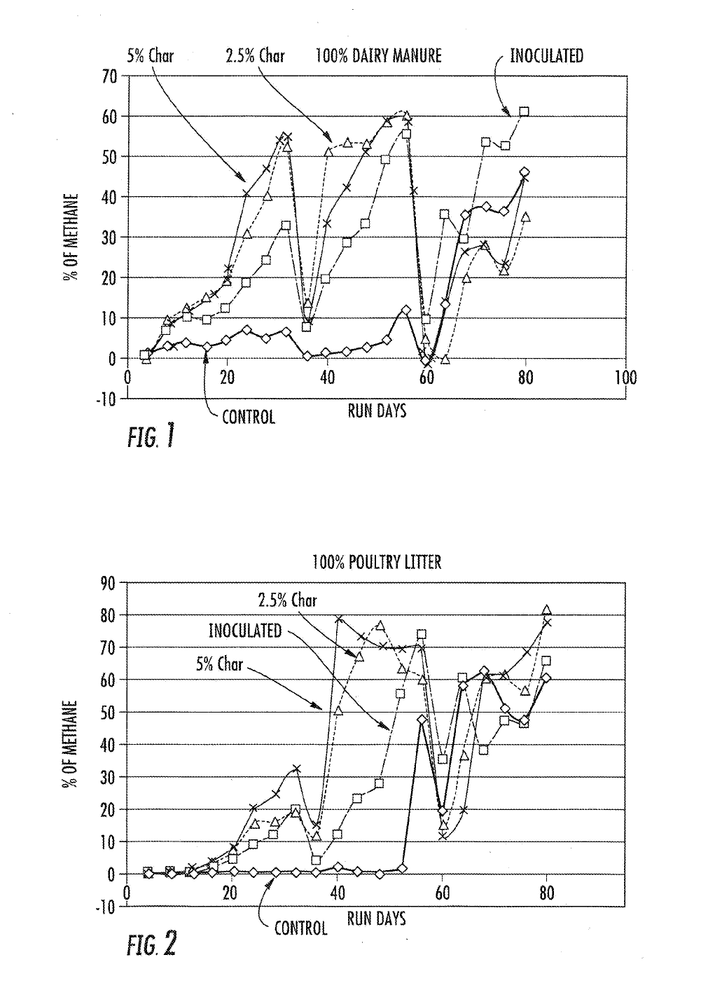

Methane production in treatments having 100% Dairy Manure was better with 2.5% of char. However, in 100% poultry litter, 5% char recorded highest methane production. But the difference was not significant between 2.5% and 5% char in 100% poultry litter as substrate. The cumulative methane production in the reactor containing 100% dairy manure and 100% poultry litter is given in FIGS. 1 and 2. Treatments with 2.5% char addition showed 471% more methane production than the control in 100% Dairy manure treatment. It is evident that the impact of the char addition is better in dairy manure as the substrate. The figures also show that the addition of biochar improved the methane concentration to 55% within 35 days in dairy manure and 42 days in poultry litter. These results confirm biochar at 2.5 and 5% concentration stimulates biogas production and increase methane concentration with a short HRT.

Chemical oxygen demand (COD) removal rates for 100% PL in treatments with inoculum only, 2.5 and 5% char were 71.3, 77.9 and 87.3%, respectively. Similarly 2.5 and 5% char addition recorded 99.5 and 99.4% COD reduction in co-digestion treatments with 50% PL and 50% DM, respectively, when compared to 84.9% reduction in the treatment with inoculum only. These results clearly indicate that the addition of biochar can enhance microbial activity in the digester, accelerate biodegradation of organic pollutants and result in maximum COD removal (e.g., COD reduction of about 40 to 100%, about 70 to 99.9%, about 87 to 99.9%, 40% or more, 70% or more, 84% or more, about 90% or more, or 99% or more) to increase energy recovery in the form of methane.

TABLE 1
Effect of char addition on COD removal

| | | | | COD |
|---|---|---|---|---|
| | | | COD | reduction |
| Control | Initial COD | Final COD | consumed | (%) |
| 100% PL | 27,500 | 14,700 | 12,800 | 46.5 |
| 50% PL + 50% DM | 21,500 | 10,800 | 10,700 | 49.8 |
| 100% DM | 8,700 | 5,100 | 3,600 | 41.4 |
| Inoculum only | | | | |
| 100% PL | 35,500 | 10,200 | 25,300 | 71.3 |
| 50% PL + 50% DM | 23,800 | 3,600 | 20,200 | 84.9 |
| 100% DM | 8,000 | 100 | 7,900 | 98.8 |
| 2.5% Char | | | | |
| 100% PL | 31,200 | 6,900 | 24,300 | 77.9 |
| 50% PL + 50% DM | 20,000 | 100 | 19,900 | 99.5 |
| 100% DM | 5,200 | 100 | 5,100 | 98.1 |
| 5% Char | | | | |
| 100% PL | 31,600 | 4,000 | 27,600 | 87.3 |
| 50% PL + 50% DM | 15,600 | 100 | 15,500 | 99.4 |
| 100% DM | 22,700 | 100 | 22,600 | 99.6 |

### Example 2

This example describes the evaluation of the co-digestion of dairy manure (DM) and algal biomass (AB) in varying combinations and study the stimulatory effects of algae and addition of pyrolized pine char on methane production.

### Methodology

Five different mixtures of dairy manure and algae biomass (100% DM, 75% DM+25% AB, 50% DM+50% AB, 25% DM+75% AB and 100% AB) were used for the study and the experiments were carried out in 2 L batch reactors. An active anaerobic inoculum prepared using DM with rumen fluid was used as inoculum (20% WN) for all the treatments. Addition of 2.5 and 5% (WN) of granular pyrolized pine char were used as a stimulant for all combinations of dairy manure and algae. Biogas production was measured three times per week for 55 days using water displacement technique. Methane content was analyzed using a GC.

### Results

Total Biogas production from these batch reactors was 14.7 L, 13.4 L and 8 L for the reactors added with 2.5 and 5% char and inoculum without char in the treatments containing 75% DM+25% AB, respectively, whereas the control showed only 2.9 L. Maximum methane concentration observed was 81.15%, 76.72% and 67.71% for the reactors added with 5 and 2.5% char and inoculum without char, respectively, in the treatments containing 75% DM+25% AB, whereas the control treatments showed a maximum methane concentration of 7.85% only. Addition of 2.5% char showed 83.75% increase in total biogas production in comparison with the treatment without char addition. Enhancing biogas production with addition of 2.5% granular pine char is an innovative approach for biomethane production using livestock and biomass wastes.

### Example 3

This example describes the effect of biochar addition (5, 7 and 10% levels) in a fed-batch anaerobic digestion process using 75% dairy manure+25% algae mix as feedstock

### Methods

Dairy manure and algal biomass were used as feedstocks for the co-digestion experiments. Dairy manure was obtained from the UGA teaching dairy farm. Spirulina algal biomass used in the co-digestion study was obtained from Earthrise farms, California. The pine char used in the experiment as biostimulant was obtained from the UGA Pyrolysis lab. Rumen fluid collected from dairy cows was used as inoculum after filtration.

The experiment was planned to test the effect of biochar addition at 5, 7, and 10% levels in 75% dairy manure+25% algae mix. Experiment was conducted in 2 L capacity reactors with butyl rubber stoppers in fed-batch mode. The working volume of the digesters was 500 mL. Every week 50 mL of the reactor content without biochar was removed and premixed feedstock for each treatment was added 15 times at the rate of 50 mL each week in order to have a working volume of 500 mL. After adding the required amounts of inoculum (10% v/v) and substrate (s), each digester was purged with nitrogen gas for 5 min to assure anaerobic condition before it was tightly closed with a rubber stopper. In each experimental run, the biogas production from three blank digesters that contained the same amount of inoculum and water was measured. All the digesters were manually shaken once a day for 1 min.

The daily biogas production of each digester was determined by the volume of biogas produced through water displacement. Biogas samples were taken every day and analyzed for the contents of methane (CH₄) and carbon dioxide (CO₂) using a GC (Hewlett Packard 5890A, USA) equipped with a thermal conductivity detector every three days. Total solid (TS), VS and VSS were determined in the well-mixed samples in triplicates according to the standard methods (APHA, 1998). The carbon-nitrogen content was analyzed using the LECO 932 equipment. Chemical oxygen demand (COD) was estimated using HACH equipment and reagents for HR (0-1500 mg/L) based on the dichromate method. The volatile fatty acids (VFAs) were quantified using GC and flame ionization detector (FID). pH was monitored with help of pH strips. Total and volatile solids were estimated using the Leco proximate analyzer TGA-701.

### Results

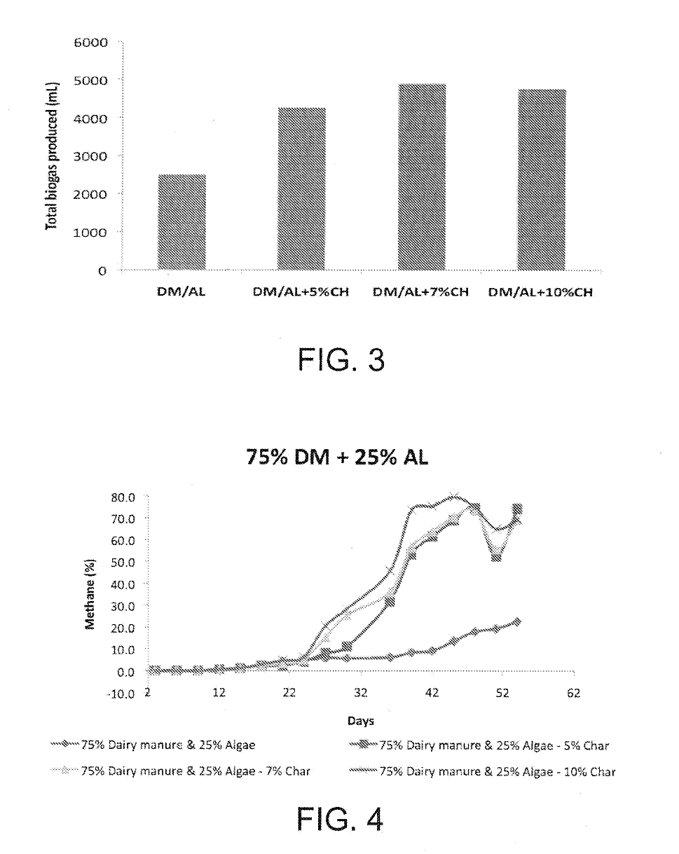

Compared to all the treatments the co-digestion treatment containing 75% dairy manure+25% algae with 7% biochar recorded highest biogas production (4.89 L) followed by the treatment added with 10% biochar (4.76 L) and 5% biochar (4.27 L) (FIG. 3). Although it was lesser than the treatments containing 7 and 10% char, the treatment added with 5% biochar also produced more biogas when compared to the control without the addition of char. The treatment with 10% char recorded 70% methane concentration on 42^{nd} day.

Treatments containing 7 and 10% char recorded 70% methane concentration on 52^{nd} day (FIG. 4).

### Discussion of Examples 1 to 3

We conducted experiments to assess biochar (a by-product of pyrolysis process derived from plant biomass) as a low-cost biostimulant to enhance biogas production, methane concentration and COD degradation. Various feedstocks such as Dairy manure, Poultry litter, Algae biomass, and a combination of 75% dairy manure+25% algae were used in the studies. Various levels of biochar addition viz. 2.5, 5, 7 and 10% were tested at batch and fed-batch mode. The findings are given below:
2.5% biochar addition recorded highest methane production (471% increase than control) in dairy manure.
5% biochar addition recorded highest methane production in Poultry litter
55% concentration of methane in biogas was achieved within a short HRT in dairy manure (within 35 days) and poultry litter (42 days) in the treatments added with 2.5 and 5% char.
Biochar addition enhanced microbial activity and significantly increased the rate of COD degradation in 100% PL and treatments containing 50% PL+50% DM.
2.5% biochar addition significantly improved biogas and methane production in the batch co-digestion treatments containing 75% DM+25% algae.
10% biochar addition recorded highest biogas production and reduced the HRT in the fed-batch co-digestion treatment containing 75% DM+25% algae.

These results confirm that biochar of the present disclosure can be used as a biostimulant and it can be used to enhance biogas and methane production from various degradable organic feedstocks. Biochar adsorbs and reduces the impact of toxic components present in the feedstocks (e.g., ammonia) and offers protection to hydrolytic and methanogenic bacteria. Also biochar acts as a support growth medium for anaerobes. Embodiments of the present disclosure provide for the development of low-cost anaerobic digesters with methanogens immobilized in biochar for efficient recovery of methane from agricultural, industrial and municipal wastes. It will help dairy, poultry and swine farms to effectively manage their wastes and generate income from waste management.

### Example 4

### Immobilization of a Methanogenic Consortium on Biochar Support

### Part A—Identification of Time of Incubation to Immobilize Bacteria on Biochar

Bacterial colonization of the biochars was examined in Part A. Biochar was created from pine char and rumen fluid (from the rumen of a cow), which was used as inoculums.

Ten polyester bags containing 1 g of pine char each one were placed in a 1-L Erlenmeyer flask. Inoculum of 100 mL was placed in the flask without allowing the entry of air/oxygen. Over 32 days the reactors were shaken at 90 rpm at maintained at 37° C. Once every three days a bag was sampled and stored at −20° C. to measure protein content and take SEM pictures. Methane and biogas were not measured during this experiment. Bradford method was used to test the protein content. Cellular lysis of the bacteria attached to char was done as follows: 0.5 g of immobilized inocula on char was placed in a 1.5 mL microtube and 1 mL of water was added. Samples were centrifuged at 5,000 rpm for 10 min to allow the precipitation of char but no unattached cells. The supernatant was discarded and 1 mL of NaOH 1N was added to the pellet. The sample was placed in a boiling water bath for 20 min and subsequently centrifuged at 10,000 rpm for 15 min. The supernatant was recovered to measure the protein content. The sample was stored at 4° C. until use.

### Results

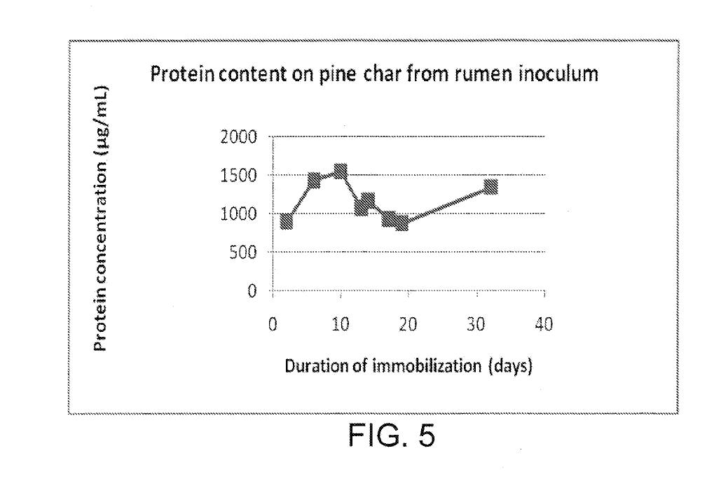

Samples obtained on the 10^{th} day shows the highest content of protein (See FIG. 5). From the start, there is a steady increase in protein content, indicating higher levels of colonization. After the 10^{th} day there is a decline that appears to be rectified past the 20^{th} day.

Part B—Evaluation of Peanut Hull Biochar Immobilize with Bacteria on Biogas Production Performance.

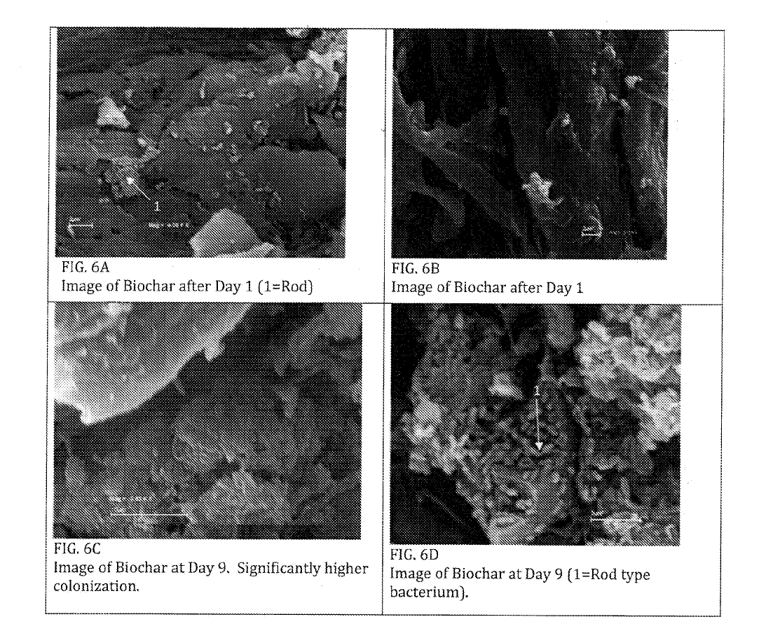

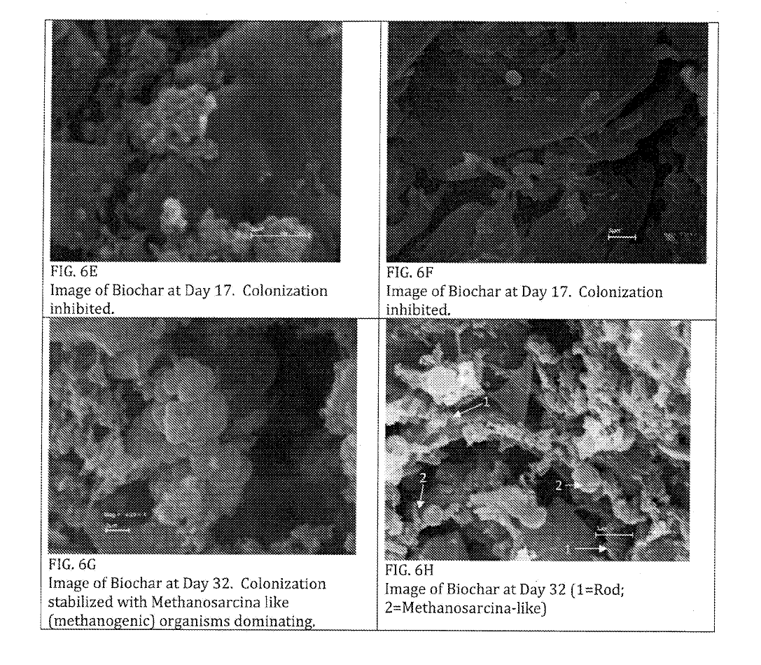

The following describes the performance of bacterial immobilized biochar as inoculums was compared with adding direct liquid inoculum. FIGS. 6A to 6F illustrate scanning electron microscope pictures that show morphologies found on pine char surface at different incubation time.

### Method

Peanut hull biochar was impregnated with rumen inoculum for 10 days and added to a synthetic feedstock at 7% solids content and C:N ratio of 30:1. Inocula were tested using sterile and non-sterile feedstock conditions. Treatments (biochar based microbial carriers) were compared against control that included liquid inoculum at 10% by volume. Working volume of the test reactors was 50 mL contained in a 100 mL serum bottle. Bottles were sealed with rubber caps and aluminum crimpers. Head space was filled with N₂ to initiate anaerobic conditions and biogas produced was released daily and biogas volume measured.

### Results

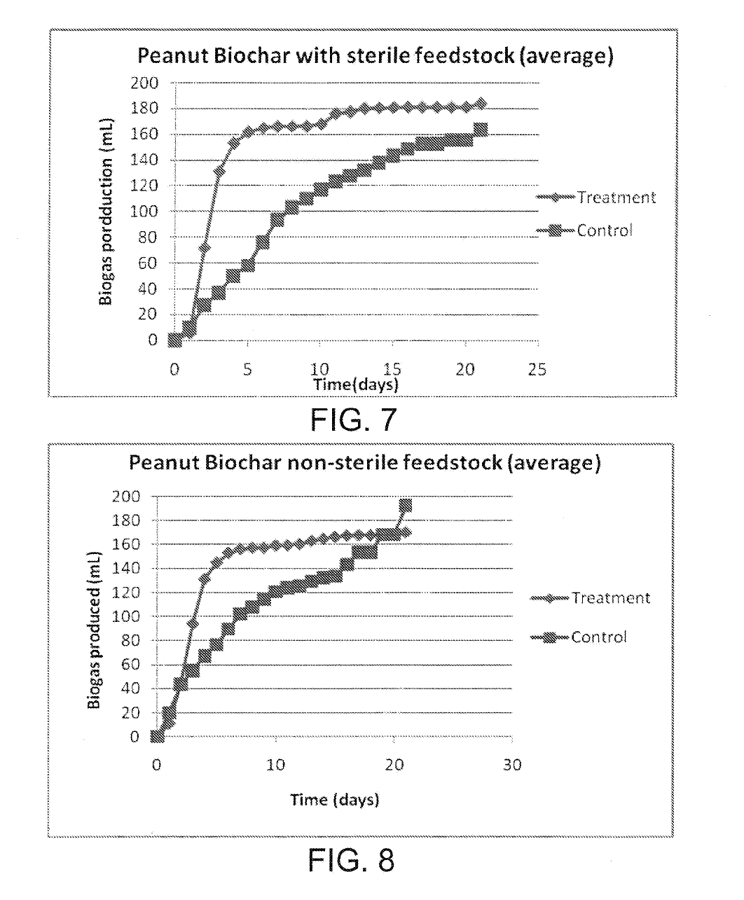

Peanut hull biochar immobilized with inoculum reached biogas production of 162 mL by the 5^{th} day, where controls reached that value only on the 21^{st} day (See FIG. 7). Total biogas production in the treatment was 183.7 mL relative to 165.7 mL in the control.

When using non-sterile feedstock, the treatment with biochar immobilized bacteria produced 153.0 mL biogas by the 6^{th} day, while it took the control 17 days to produce 153.7 mL biogas (See FIG. 8). Both data sets are averages of three replicates and show that using biochar impregnated inoculum can reduce the biogas production time from about 21 days (in the case of liquid inoculum added controls) down to about 5 to 6 days.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

We claim:

1. A material comprising:
a biochar impregnated with at least one type of microbe.

2. The material of claim 1, wherein the microbe includes a methanogen type of microbe.

3. The material of claim 1, wherein the type of microbe is selected from the group consisting of: algae, fungi, bacteria, archaea, protist, and a combinaton thereof.

4. The material of claim 1, wherein the biochar is a carbonized plant material.

5. The material of claim 1, wherein the microbe is bacteria.

6. A method of producing a gas, comprising:
exposing a biochar of claim 1 to a material selected from the group consisting of: a biomass, manure, and a combination thereof; and
producing a gas from the interaction of the material with the microbes.

7. The method of claim 6, wherein the manure is a material selected from the group consisting of: poultry litter, dairy manure, and a combination thereof.

8. The method claim 6, wherein the material includes both biomass and manure.

9. The method of claim 6, wherein the biomass includes algal biomass.

10. The method of claim 6, wherein the gas is methane.

11. A reactor for producing a gas, comprising a material of claim 1.

12. The reactor of claim 11, comprising an anaeorobic digester.

13. The method of claim 12, wherein the gas is methane.
